(19) United States
(12) Patent Application Publication
(10) Pub. No.: US 2013/0116745 A1
(43) Pub. Date: May 9, 2013

(54) NEUROSTIMULATOR SYSTEM, APPARATUS, AND METHOD

(71) Applicants: Kellie Fletcher, Oakland, CA (US); Patrick Hard, Redwood City, CA (US); Anthony V. Caparso, San Francisco, CA (US); Karen Tsuei, Redwood City, CA (US)

(72) Inventors: Kellie Fletcher, Oakland, CA (US); Patrick Hard, Redwood City, CA (US); Anthony V. Caparso, San Francisco, CA (US); Karen Tsuei, Redwood City, CA (US)

(73) Assignee: AUTONOMIC TECHNOLOGIES, INC., Redwood City, CA (US)

(21) Appl. No.: 13/722,260

(22) Filed: Dec. 20, 2012

Related U.S. Application Data
(63) Continuation-in-part of application No. 12/688,524, filed on Jan. 15, 2010.
Application No. 12/688,524 is a continuation-in-part of application No. 12/765,712, filed on Apr. 22, 2010.
(60) Provisional application No. 61/578,337, filed on Dec. 21, 2011.
(60) Provisional application No. 61/145,003, filed on Jan. 15, 2009.
(60) Provisional application No. 61/171,749, filed on Apr. 22, 2009.
(60) Provisional application No. 61/177,895, filed on May 13, 2009.

(51) Int. Cl. A61N 1/36

(52) U.S. Cl. CPC A61N 1/36075
USPC 607/46

(57) ABSTRACT

The invention relates to a method (200) for conducting a clinical trial with a medical device. The method (200) includes the step (202) of providing a medical device to a trial subject, the medical device being capable of providing an adjustable level of therapy. The method also comprises the step (204) of using the medical device to apply therapy to the trial subject. The method further comprises the step (206) of controlling the application of therapy applied to the trial subject according to a clinical trial design.

### RELATED APPLICATIONS

This application is a continuation-in-part of U.S. patent application Ser. No. 12/688,524, filed Jan. 15, 2010, titled “APPROVAL PER USE IMPLANTED NEUROSTIMULATOR,” which claims the benefit of U.S. Provisional Application No. 61/145,003 filed Jan. 15, 2009. This application is also a continuation-in-part of U.S. patent application Ser. No. 12/765,712, filed Apr. 22, 2010, titled “IMPLANTABLE NEUROSTIMULATOR WITH INTEGRAL HERMETIC ELECTRONIC ENCLOSURE, CIRCUIT SUBSTRATE, MONOLYTHIC FEED-THROUGH, LEAD ASSEMBLY AND ANCHORING MECHANISM,” which claims the benefit of U.S. Provisional Application No. 61/171,749 filed Apr. 22, 2009, and U.S. Provisional Application No. 61/177,895 filed May 13, 2009. The full disclosures of these applications are hereby incorporated by reference in their entirety.

### FIELD OF THE INVENTION

The invention relates generally to systems, devices, and methods for using an implantable medical device to deliver therapy to a patient. More specifically, according to one aspect of the invention, systems, devices, and methods according to the invention are used to deliver electrical stimulation to a peripheral, central or autonomic neural structure. In one particular aspect, the invention relates to neurostimulator systems, devices, and methods for treating primary headaches, such as migraines, cluster headaches, trigeminal autonomic cephalalgias and/or many other neurological disorders, such as atypical facial pain and/or trigeminal neuralgias.

### BACKGROUND OF THE INVENTION

Primary headaches are debilitating ailments that afflict millions of individuals worldwide. The specific pathophysiology of primary headaches is not known. Known causes of headache pain include trauma, vascular defects, autoimmune deficiencies, degenerative conditions, infections, drug and medication-induced causes, inflammation, neoplastic conditions, metabolic-endocrine conditions, iatrogenic conditions, musculoskeletal conditions, and myofacial causes. In many situations, however, even though the underlying cause of the headache may be identified and treated, the headache pain itself may persist.

Recent clinical studies in treatment of headaches have targeted the manipulation of sphenopalatine (pterygopalatine) ganglion (SPG), a large, extra cranial parasympathetic ganglion. A ganglion is a mass of nervous tissue found in some peripheral and autonomic nerves. Ganglia are located on the roots of the spinal nerves and on the roots of the trigeminal nerve. Ganglia are also located on the facial, glossopharyngeal, vagus and vestibulochoclear nerves. The SPG is a complex neural ganglion with multiple connections, including autonomic, sensory, and motor connections. The SPG includes parasympathetic neurons that innervate, in part, the middle cerebral and anterior cerebral blood vessels, the facial blood vessels, and the lacrimal glands.

The maxillary branch of the trigeminal nerve and the nerve of the pterygoid canal (also known as the vidian nerve which is formed by the greater and deep petrosal nerves) send neural projections to the SPG. The fine branches from the maxillary nerve (pterygopalatine nerves) form the sensory component of the SPG. These nerve fibers pass through the SPG and do not synapse. The greater petrosal nerve carries the preganglionic parasympathetic axons from the superior salivary nucleus, located in the Pons, to the SPG. These fibers synapse onto the postganglionic neurons within the SPG. The deep petrosal nerve connects the superior cervical sympathetic ganglion to the SPG and carries postganglionic sympathetic axons that again pass through the SPG without any synapses.

The SPG is located within the pterygopalatine fossa. The pterygopalatine fossa is bounded anteriorly by the maxilla, posteriorly by the medial plate of the pterygoid process and greater wing of the sphenoid process, medially by the palatine bone, and superiorly by the body of the sphenoid process. The lateral border of the pterygopalatine fossa is the pterygomaxillary fissure, which opens to the infratemporal fossa.

Various clinical approaches have been used to modulate the function of the SPG in order to treat headaches, such as cluster headaches or chronic migraines. These approaches vary from lesser or minimally invasive procedures (e.g., transnasal anesthetic blocks) to procedures or greater invasiveness (e.g., surgical ganglionectomy). Other procedures of varying invasiveness include those such as surgical anesthetic injections, ablations, gamma knife procedures, and cryogenic surgery. Although most of these procedures can exhibit some short term efficacy in the order of days to months, the results are usually temporary and the headache pain eventually reoccurs.

Ongoing clinical studies evaluate the efficacy, safety, and reliability of new devices and methods for treatment of such headaches. A proposed new therapy requires approval from a regulatory authority before being granted permission to be marketed. Clinical studies normally include prospectively randomized controlled studies, where patients are randomly assigned to control and experimental groups. Such studies typically include a patient group which receives a placebo treatment, as for example when the study relates to a new pharmacological agent. Placebo therapies may be desirable to in order to study the efficacy, safety, reliability, and reproducibility of the proposed therapy during a clinical trial.

In the case of new drugs a patient may be given a placebo compound, making them a control subject. In the case of device therapy, producing a control subject is much more difficult. For example, if the device therapy involves delivery of stimulation that the patient can sense through feel, it would be difficult to produce a control subject, due to the need to produce the sensed feel without providing the stimulation. As such, it can be a challenge to conduct clinical studies of medical devices where patients receiving stimulation from the devices are compared to a control group that does not receive the stimulation.

Accordingly, there exists a great need for improved devices and methods for providing longer term efficacy in patients suffering from headaches and, in particular, methods and devices for conducting clinical studies involving medical devices that provide treatments via stimulation.

### SUMMARY OF THE INVENTION

The invention relates to systems, devices, and methods for using an implantable medical device (“IMD”) to deliver therapy to a patient. According to one aspect, the invention relates to an IMD for delivering electrical stimulation to a peripheral, central or autonomic neural structure. In this aspect, the IMD may comprise a neurostimulator for treating primary headaches, such as migraines, cluster headaches, trigeminal autonomic cephalalgias and/or many other neurological disorders, such as atypical facial pain and/or trigeminal neuralgias.

According to one aspect, the devices, systems, and methods enable a patient to respond to a therapy regimen during a clinical trial evaluation, so that the efficacy of the therapy regimen can be determined and recorded. The devices, systems, and methods may also enable the evaluation of the patient's response(s), e.g., by a physician. The devices, systems, and methods, further may be useful in treatment of patients in a post-market usage after the clinical trial.

In one embodiment, an IMD and an associated handheld remote controller (“RC”) each may have an operating memory for storing a programmable operating instructions and data, both input and recorded, that govern the operation of each respective device. The IMD and RC each may also include processing hardware, associated with the operating memory, for executing the programmable operating instructions in accordance with the input and recorded data. According to one aspect, the IMD may receive from the RC operating instructions, data, or both operating instructions and data, that at least partially govern the therapies applied via the IMD. The governed therapies may include either or both the clinical trial therapies and the post-market usage therapies.

The IMD administers therapy in accordance with stimulation parameters stored on the IMD. The stimulation parameters may be programmed into the IMD in a variety of manners. For example, the stimulation parameters may be programmed via a programming system, which can be either local to or remote from the device. Local programming of the IMD can be accomplished, for example, with the patient near a physician's workstation, which can communicate wirelessly with the device (e.g., via Bluetooth, long range induction, etc.) or with the RC acting as an interface or wand to the device. Remote programming of the IMD can be accomplished by establishing communication between with the device via one or more communication networks. For example, a remotely located physician's workstation can communicate with a patient's personal computer via an internet connection, which relays the stimulation parameters to the IMD via the RC acting as an interface.

According to another aspect of the invention, the RC is configured to prompt for and elicit from the patient subjective and objective data, which the patient enters via the RC. The RC records the patient input data, and also records data associated with the treatment applied by the IMD. The data can then be transmitted to the physician's workstation or possibly mobile device such as a PDA, cell phone, tablet, etc., so that the physician can use the data to verify the stimulation parameters for current therapy or to determine control or stimulation parameter adjustments for subsequent therapies or determine if the current stimulation parameters provide effective therapy to the patient.

Accordingly, the invention relates to a method for conducting a clinical trial with a medical device. The method comprises the step of providing a medical device to a trial subject, the medical device being capable of providing an adjustable level of therapy. The method also comprises the step of using the medical device to apply therapy to the trial subject. The method further comprises the step of controlling the application of therapy applied to the trial subject according to a clinical trial design.

The invention also relates to an apparatus for applying stimulation therapy to a subject in a clinical trial. The apparatus includes an implantable medical device and a remote controller for inductively powering the medical device and communicating with the medical device. The implantable medical device is adapted to store therapy settings and simulation parameters for applying the stimulation therapy. The remote controller is operative to record therapy data during the application of stimulation therapy.

The invention further relates to a system for conducting a clinical trial on a medical device. The system includes an implantable medical device for applying stimulation therapy and a remote controller which uses radio frequency (RF) for inductively powering the medical device and communicating with the medical device. The implantable medical device is configured to store therapy settings and simulation parameters for applying the stimulation therapy. The remote controller being operative to record therapy data during the application of stimulation therapy. The system also includes a communication network over which the remote controller can transmit the recorded therapy data in order to conduct the clinical trial.

### BRIEF DESCRIPTION OF THE DRAWINGS

FIG. 1 is a schematic illustration of devices that form a portion of a system for delivering therapy using an implantable medical device, according to the invention;

FIGS. 2A and 2B illustrate a portion of the system implanted in a patient;

FIG. 3 is a schematic block diagram of the system for delivering therapy using an implantable medical device, according to the invention;

FIGS. 4A-4D are schematic block diagrams of certain configurations of the system of FIG. 3, according to the invention;

FIGS. 5A-5F are functional block diagrams illustrating various steps in a process that can be performed by the system of FIGS. 1-4B, according to the invention; and

FIG. 6 is a functional block diagram illustrating various steps in a process that can be performed by the medical device of FIG. 1, according to the invention.

### DESCRIPTION OF PREFERRED EMBODIMENTS

The invention relates to systems, devices, and methods for imparting a therapy on a patient in at least one of a clinical trial and a post-trial clinical usage. More particularly, the invention relates to devices, systems, and methods for applying patient stimulation therapies to at least one of clinical trial subjects and post-market patients. According to one aspect of the invention, the devices, systems, and methods of the invention allow for conducting a clinical trial via variations in applied stimulation therapies according to a clinical trial design. In one particular example implementation, the devices, systems, and methods of the invention can be used to conduct a clinical trial according to a random insertion of placebo trial design.

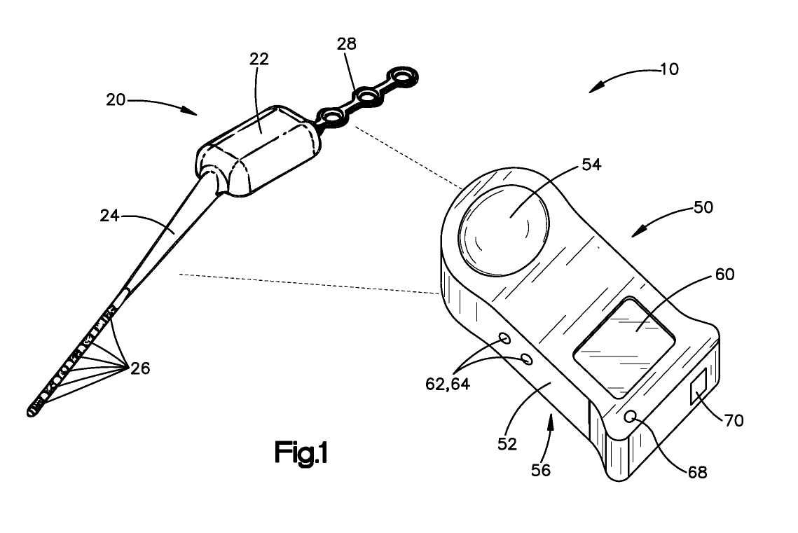

FIG. 1 illustrates by way of example a medical device forming a portion of a system that can be implemented in accordance with the invention. Referring to FIG. 1, according to one aspect of the invention, a medical device 10 includes an implantable medical device (“IMD”) 20 and a handheld remote controller (“RC”) 50 for interfacing with and controlling operation of the IMD. In this description, the term “implantable” is meant to describe that the medical device is configured for in vivo placement in the patient by surgical or other means. In the illustrated embodiment, the IMD 20 is shown and described as an implantable neurostimulator. The IMD 20 may, for example, be a neurostimulator of the type shown and described in co-pending U.S. Patent Application Publication No. US 2010/027413 A1, the disclosure of which is hereby incorporated by reference in its entirety.

The neurostimulator embodying the IMD 20 illustrated and described herein is an example of just one particular IMD that may be implemented in accordance with the systems, devices, and methods of the invention. Those skilled in the art will appreciate that the systems, devices, and methods of the invention can be applied to implantable stimulators other than the neurostimulator illustrated in FIG. 1 without departing from the spirit of the invention. Those skilled in the art will also appreciate that the systems, devices, and methods of the invention can be applied to IMDs other than stimulators, in general, and other than neurostimulators, more specifically.

The IMD 20 illustrated in FIG. 1 includes a stimulator body 22, a stimulator lead 24 including one or more stimulating electrodes 26, and an anchoring portion 28. The IMD 20 is an inductively powered system having stored programmed stimulation parameters and bi-directional telemetry to facilitate communication between the implanted device and the external RC 50. The body 22 comprises an electronics enclosure that can house, for example, an application-specific integrated circuit, various passive components, and an antenna/coil for radio frequency transfer of power and communication. The lead 24 provides an electrical connection between the electronics housed in the body portion 22 and the stimulating electrodes 26. Each of the one or more electrodes 26 provides a site for electrical stimulation of the target anatomy.

The IMD 20 of the example embodiment of FIG. 1 is powered inductively by the RC 50 and has electronics, micro-electronic components, and integrated circuits necessary to store settings, parameters, and other data. For example, in an embodiment used in a clinical trial, the IMD 20 may store programmable stimulation parameters and clinical trial specific settings. In another example, in an embodiment used in a post-market patient usage, the IMD 20 may store programmable stimulation parameters and post-market specific settings.

During a therapy session, the IMD 20, powered by the RC 50, delivers electrical stimulation per the stimulation parameters stored on the IMD. Each of the electrodes 26 is controllable independently, which allows the physician to select which electrodes will serve as anodes and which electrodes will serve as cathodes in any combination. The IMD 20 can apply the stimulation therapy in accordance with the stimulation parameters stored on the IMD. Additionally, the IMD 20 can acquire and transmit to the RC 50 therapy session data gathered during a therapy session. The IMD 20 includes a non-volatile memory for storing the stimulation parameters and other clinical trial related information.

The RC 50 provides inductive power to the IMD 20 and communicates (e.g., via radio frequency) with the IMD. Through this communication, the RC 50 can access settings and parameters stored on the IMD 20 and also record therapy session data (in real-time or at a predetermined time). For example, in an embodiment used in a clinical trial, the RC 50 may record therapy session data in real time while also accessing clinical trial specific data (e.g., trial type, specific questions to be asked, therapy randomization strings, language settings, etc.) in real-time or at some predetermined time before, during, or after the therapy session. In another example, in an embodiment used in a post-market patient usage, the RC 50 may record therapy session data in real time while also recording post-market specific data (e.g., specific questions to be asked) in real-time or at some predetermined time before, during, or after the therapy session.

In this description, reference is made to “patients” and “subjects” interchangeably. The term “patients” can be used to describe patients in a clinical trial setting or in a post-market usage. Similarly, the term “subjects” can be used to describe patients in a post-market usage or in a clinical trial setting.

Also, in this description, reference is made to stimulation parameters that may be programmed and/or stored on the IMD 20 and accessed and transmitted to/from the RC 50. The term “stimulation parameters,” as used herein, is meant to encompass the parameters that define the stimulation therapy applied to the patient by the medical device 10. In a clinical trial setting, the stimulation parameters may include parameters for each of several therapy modes or configurations used during the clinical trial. The stimulation parameters include, but are not limited to, the parameters set forth below in Table 1:

TABLE 1
Stimulation Parameters
Amplitude
Frequency
Pulse Width
Pulse Interval
Electrode Settings
Patient Limits
Amplitude Ramp Rates
Pulse Width Ramp Rates
Biphasic/Monophasic Stimulation Pulses

Additionally, in this description, reference will be made to therapy settings and clinical trial settings programmed and/or stored on the IMD 20 and accessed and transmitted to/from the RC 50. The term “therapy settings,” as used herein, is meant to refer to patient specific settings that customize the medical device 10 according to patient needs/preferences and physician/clinician requirements. The therapy settings may include post-market patient usage settings, clinical trial settings, or a combination of both. The term “clinical trial settings,” as used herein, is meant to refer to settings and/or data related to a clinical trial conducted using the medical device 10. The therapy/clinical trial settings include, but are not limited to, the settings set forth below in Table 2:

TABLE 2
Therapy/Clinical Trial Settings & Data
Language Preference
Screen Brightness, Color, etc.
Screen Appearance/Fonts
Text Size
Volume
Patient Diary Questions
Diary Date/Time Schedule
Clinical Trial Therapy Mode Data
Trial Randomization Strings
Clinical Trial Type
Therapy Mode Data
Trial Subject Diary Entries
Diary Dates/Times
Trial Randomization Strings
Trial Type
Hardware Identification/Ser. No.

Additionally, in this description, reference will be made to therapy session data that can be recorded, stored and transmitted by the RC 50. The term “therapy session data,” as used herein, is meant to refer to data related to the therapy applied by the medical device 10 during a session. The therapy session data includes, but is not limited to, the data set forth below in Table 3:

TABLE 3
Therapy Session Data
Electrode Impedances
Electrode Opens/Shorts
Date/Time of Therapy Start
Date/Time of Therapy End
Therapy Session Duration
Manual Patient Adjustments
Patient Attempted Adjustments
Ramp Times
Maximum Amplitude
Actual Amplitudes
Time at Each Setting
Trial Subject Diary Entries
Diary Dates/Times
Software/Firmware Versions
Serial Number Data
Data Integrity Check Data
IMD Register Values
Tilt/Fault Data
Communication Loss Data

Referring to FIG. 1, the RC 50 includes a body portion 52 and an antenna portion 54. The body portion 52 is ergonomically shaped/contoured and includes a graphical user interface 56 that includes a display 60 and a user navigation/input controls 62. The display 60 may, for example, comprise an LED or LCD display, color or B&W, that displays the status, settings, and other data related to the operation of the RC 50. The RC 50 interface 56 may be fully customizable, i.e., the color, brightness, font, text size, sound volume, etc. can be adjusted to suit the patient. The user navigation/input device 62 illustrated in FIG. 1 comprises control buttons 64 that are located at an ergonomic position on the RC 50 so that they can be accessed with either right or left hand while the controller is held in the therapy position. The user navigation/input device 62 could, however, comprise alternative devices, such as a touch screen, track ball, touch pad, thumb wheel, etc. The navigation/input device 62 may also be customizable in that the buttons and other devices may have functions that are programmable or assignable by the user.

The RC 50 also includes a power button 68 and I/O ports in the form of a USB/charging port 70. The RC 50 may include multiple ports and other connectivity features for providing flexibility in communications, data transfer, software/firmware uploading/downloading. Such additional ports may also be used to provide for connectivity and expandability with peripheral devices, such as a Bluetooth or other add-on communications module, or a remote inductive coil device.

The RC 50 includes a power source that includes batteries (disposable or rechargeable) and may also be powered externally via a cable connection (e.g., via AC outlet or USB). The antenna portion 54 includes a coil for inductively powering the IMD 20 and for communicating with the IMD. The RC 50 is configured to power and communicate with the IMD 20 when the RC 50 is brought into a predefined proximity (e.g., within 5 centimeters) of the IMD.

The RC 50 is illustrative of one example controller that may be implemented in accordance with the systems, apparatuses, and methods of the invention. Those skilled in the art will appreciate that certain aspects of the systems, apparatuses, and methods of the invention can be applied to controllers other than the RC 50 illustrated in FIG. 1 without departing from the spirit of the invention.

In operation, the RC 50 transfers energy to the IMD 20 via near field electromagnetic induction. The RC 50 transmits power signals via the power antenna, located in the antenna portion 54, at a specific frequency. The IMD 20 includes a power coil/antenna that is tuned to resonate close to the frequency at which the RC 50 transmits the power signal and thereby generates, through induction, power for the IMD.

The RC 50 also communicates with the IMD 20 to provide, for example, stimulation parameters, software/firmware upgrades, and other operating instructions and data prior to or subsequent to IMD implantation. The RC 50 may also receive from the IMD 20 therapy session data, handshaking communications, and current stimulation parameter settings.

The antenna(s) employed in the antenna portion 54 of the RC 50 may have various designs. For example, the antenna portion 54 may include a first antenna for inductively powering the IMD 20 and for transmitting communications to the IMD. The antenna portion 54 may also include a second antenna for receiving communication transmissions from the IMD 20.

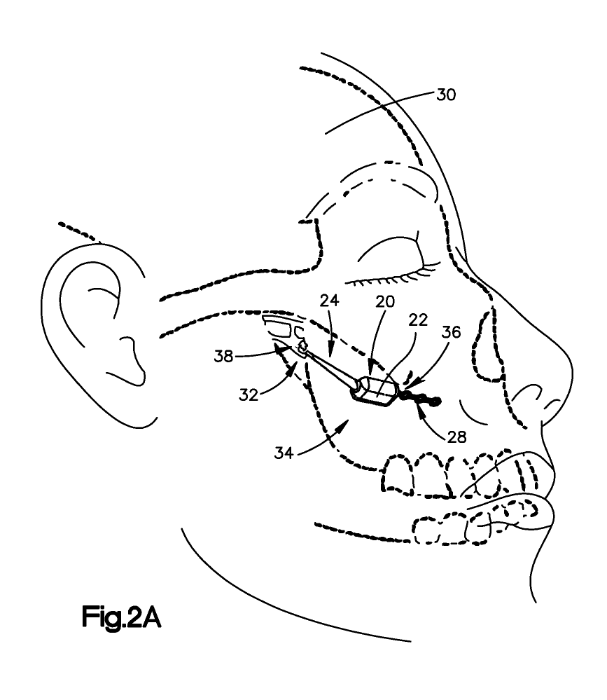

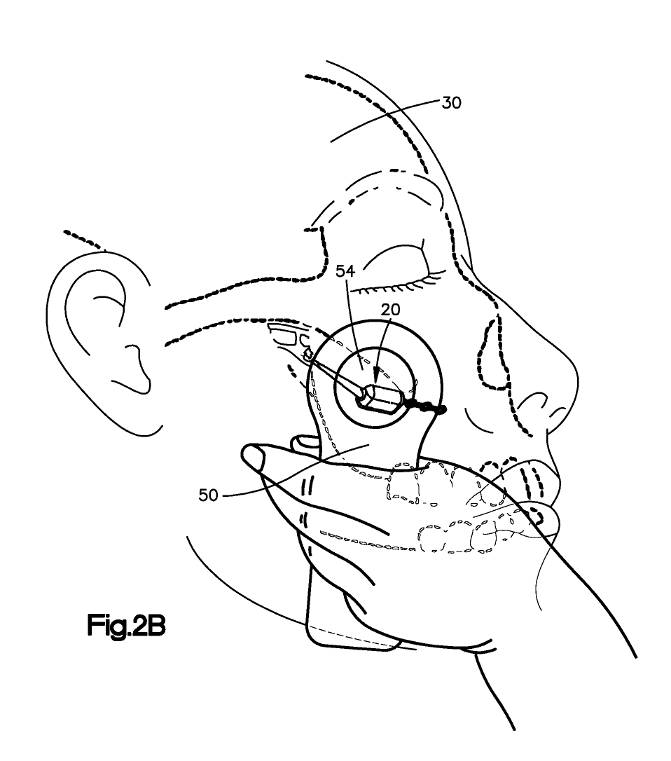

FIGS. 2A and 2B illustrate, by way of example, an implementation of the system 10 in which the IMD 20 is implanted in a patient in order to treat primary headaches, such as migraines, or other neurological disorders. As illustrated in FIG. 2A, the IMD 20 is implantable in a patient's head 30. In this particular implementation, the IMD 20 is implanted such that the stimulator body 22 is positioned medial to the zygoma 32 on the lateral/posterior maxilla 34 within the buccal fat pad of the cheek, and the integral fixation apparatus 28 is anchored to the zygomaticomaxillary buttress 36, such as by using standard craniomaxillofacial bone screws, for example. The stimulation lead 26 can be placed within the pterygopalatine fossa or, more specifically, in very close proximity to the sphenopalatine (pterygopalatine) ganglion (SPG) ganglion 38.

Referring to FIG. 2B, to operate the IMD 20, the patient manually positions the RC 50 adjacent his/her head 30 so that the antenna portion 54 is positioned in the proximity of the IMD 20. The patient can manipulate the position of the RC 50 to achieve a strong inductive link between the controller and the IMD 20. To facilitate this, the RC 50 may be configured to provide feedback in the form of sound, vibration, visual feedback, in any combination, that is indicative of the communication signal strength between the RC and the IMD 20.

Once a communication and power link is established, the IMD 20 administers the therapy in accordance with the instructions of the RC 50 and the stimulation parameters stored on the IMD. During use, the patient may be able to adjust certain stimulation parameters (amplitude, pulse width, frequency, combination thereof, or specific protocols with autonomic preset adjustment in two or more parameters at once) or parameters (ramp time, etc.) via the input device 62, e.g., by manipulating the control buttons 64. These adjustments are physician/clinician approved & configurable.

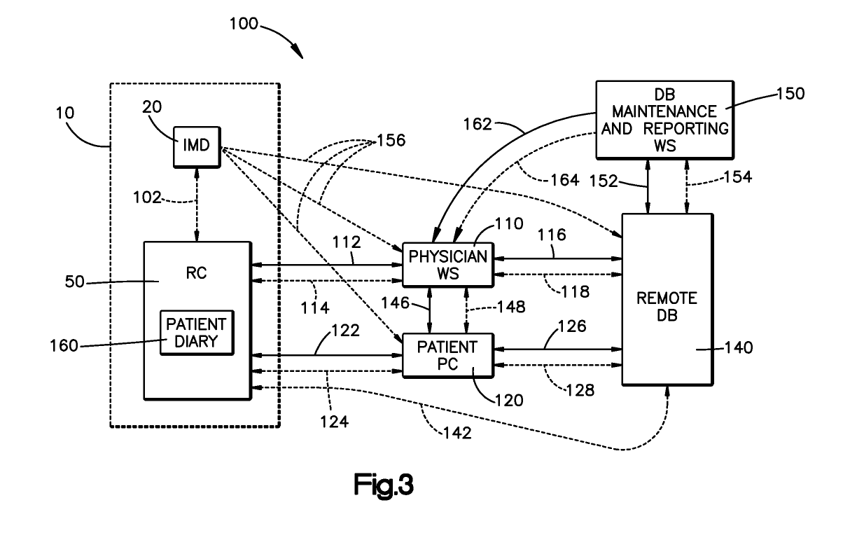

The RC 50 and IMD 20 are components of a system for applying stimulation therapy to a patient. Referring to FIG. 3, an example of a system 100 in which the RC 50 and IMD 20 are implemented includes multiple computer platforms, each of which may have a different remote location. These multiple platforms can be networked for communication with each other via a variety of wired (indicated in solid lines) and wireless (indicated in dashed lines) connections. The example system of FIG. 3 includes the medical device 10 (i.e., the IMD 20 and the RC 50), a physician workstation (“physician WS”) 110, a patient personal computer/docking station (“patient PC”) 120, and a remote database (“remote DB”) 140.

The wireless communication/power connection between the RC 50 and the IMD 20 is illustrated in dashed lines at 102. This connection 102 may, for example, employ a medical implant specific communication protocol, such as a medical implant communication system (MICS) protocol. This specialized protocol helps institute a degree of safety and reliability in communications between the RC 50 and the IMD 20, especially while the IMD is implanted in the patient.

The physician WS 110, patient PC 120, and remote DB 140 can be interconnected via a communication network 170 that includes wired connections (e.g., a wired internet connection), indicated generally with solid lines and wireless connections (e.g., a WiFi internet connection, a Bluetooth connection, or a GSM/CDMA/LTE mobile network connection), indicated generally with dashed lines. In the embodiment illustrated in FIG. 3, the physician WS 110 is has a wired connection 116 and a wireless connection 118 with the remote database 140. The patient PC 120 has a connected wired connection 126 and a wireless connection 128 with the remote database 140. The physician WS 110 and the patient PC 120 have a wired connection 146 and a wireless connection 148 with each other. The physician WS 110 and the database WS can also communicate with each other via a wired 162 or a wireless 164 connection. The physician WS 110, patient PC 120, remote DB 140, and database WS 150 can communicate with each other via the network 170 using any combination of the wired and wireless network connections.

The RC 50 can be connected to the physician WS 110 via a direct wired connection 112 (e.g., via a USB port or docking station), or via a wireless connection 114 (e.g., a WiFi connection, a Bluetooth connection, or a GSM/CDMA/LTE mobile network connection). Connected with the physician's WS 110, the RC 50 may act in a pass-through mode, allowing the physician to access the IMD 20 for programming or data retrieval via the physician WS.

The RC 50 can also be connected to the patient personal computer 120 via a direct wired connection 122 (e.g., via the USB port or docking station), or via a wireless connection 124 (e.g., a WiFi connection, a Bluetooth connection, or a GSM/CDMA/LTE mobile network connection). The RC 50 can also communicate with the remote DB 140 via the network 170. Additionally, the RC 50 can be connected directly to the remote DB 140 via a wireless connection 142 (e.g., a GSM/CDMA/LTE mobile network connection).

From the above, those skilled in the art will appreciate that the system 100 has a highly selectable configuration, and that the communication between the RC 50, physician WS 110, patient PC 120, and remote DB 140 may be configured to occur in various combinations. In this configuration, the network 170 allows for the omission of certain portions or components of the system 100 and also for redundancy in various communication channels through the network.

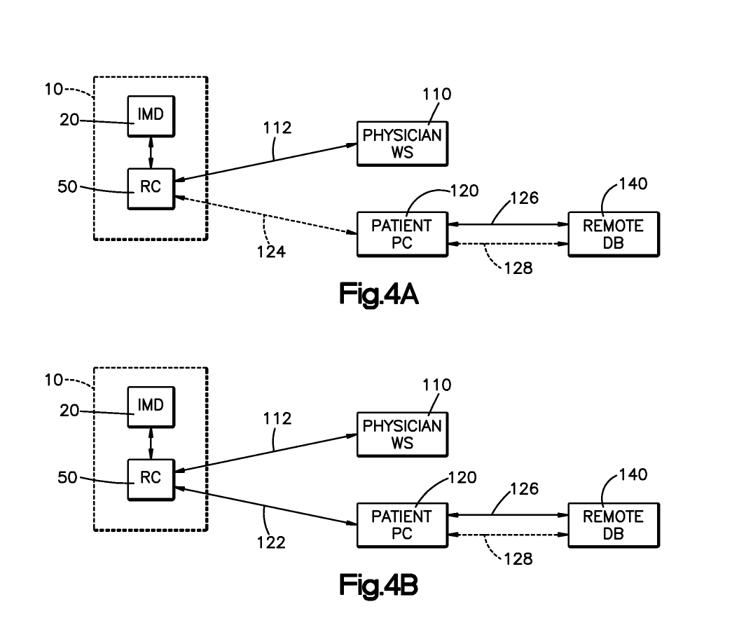

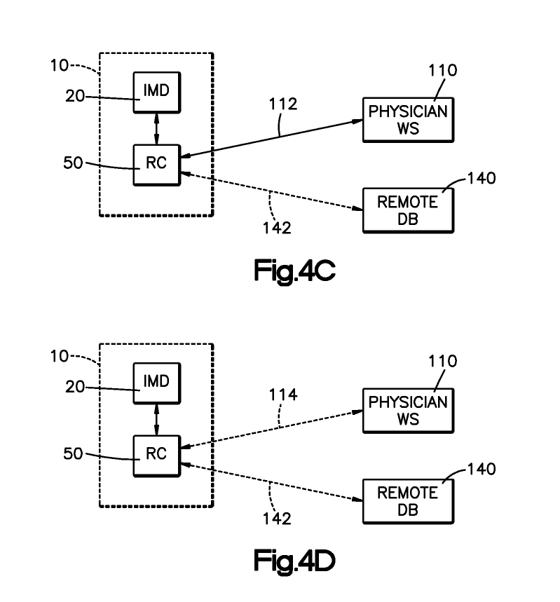

For example, referring to FIG. 4A, in one communication configuration or mode, the RC 50 may communicate with the patient PC 120 via wireless connection 124, with the physician WS 110 via wired connection 112, and with the remote DB 140 via the wireless connection with the patient PC and either the wired 126 or wireless 128 connection between the patient PC and the remote DB.

In another example communication configuration or mode, referring to FIG. 4B, the RC 50 may communicate with the physician WS 110 and with the patient PC 120 via wired connections 112 and 122, respectively, and with the remote DB 140 via the wired connection 122 and either the wired connection 126 or wireless connection 128.

In yet another example communication configuration or mode, referring to FIG. 4C, the RC 50 may communicate with the physician WS 110 via the wired connection 112 and with the remote DB 140 directly via the wireless connection 142. In this configuration, the patient PC is not necessary to establish the necessary communication channels.

In a further example communication configuration or mode, referring to FIG. 4D, the RC 50 may communicate with the physician WS 110 via the wireless connection 114 and with the remote DB 140 directly via the wireless connection 142. Again, in this configuration, the patient PC is not necessary to establish the necessary communication channels.

Referring to FIG. 3, the system 100 also includes a database maintenance and reporting workstation (“database WS”) 150 that is operatively connected to the remote DB 140 via wired 152 or wireless 154 connections. The architecture in which the database WS 150 and the remote DB 140 are implemented can vary. For example, the database WS 150 may comprise a workstation on a local area network, and the remote DB 140 may be is stored on a server in that local area network. Alternatively, the remote DB 140 may be a cloud-based database that the RC 50, patient PC 120, physician WS 110, and database WS 150 access via the internet.

The system 100 also includes a patient diary 160 in which the patient records data associated with the treatments administered via the IMD 20. The data can be in the form of responses to questions asked by the system, and the questions can be either subjective or objective in nature. The questions can be prompted and answered both prior to, during, and/or after the therapy is applied. In this manner, answering the questions may serve as a gate to patient therapy by which therapy is denied until certain diary questions are answered. Post therapy diary questions can be answered immediately after the stimulation therapy is applied or sometime thereafter. The timing and content of the questions asked both pre and post therapy can be physician/clinician selected. Post therapy diary questions can be answered via the RC 50 directly or via the patient PC 120. The patient diary can be considered a portion of the therapy/clinical trial settings & data (Table 2).

The patient diary 160 is illustrated in FIG. 3 as being part of the RC 50 because this is where the data included in the diary is entered into the system 100 by the patient. The patient diary 160 may reside in a remote location or combination of locations. For example, the patient diary 160, while collected and entered at the RC 50, may be accumulated and stored at the remote DB 140 or at the physician WS 110. As another alternative, the patient diary 160 may comprise an internet based diary stored on a remote server and accessible via the internet. As a further alternative, the patient diary 160 may comprise a cloud based system in which the diary is accessible via the internet.

Once entered, when the RC 50 is operatively connected for communication with the system 100 (wired or wirelessly), the data from the patient diary 160 can be transmitted to the remote DB 140, to the physicians WS 120, to the patient PC 110, or to a cloud based storage system. Thereafter, the physician can access the data via the workstation 110. Additionally, the patient may also be able to access certain data from the patient diary 160, such as previously answered diary questions, unanswered diary questions or additional questions, via the patient PC 120. In an internet or cloud-based implementation, the patient can access the patient diary 160 online via web access.

The questions queried to the patient for entry in the patient diary 160 can be subjective questions or objective questions. Subjective questions can serve to help describe or categorize the headache episode in terms of symptoms, severity, duration, lasting effects, etc. The data from the subjective questions in the patient diary thus give patient specific details and sensory perceptions that can be used to evaluate and adjust the therapy regimen for that particular patient. Objective questions elicit from the patient factual details not subject to the patients perception, and thus generate data that can collected along with objective data from other patients and used to evaluate efficacy for the group as a whole. The subjective and objective data collected in the patient diary includes, but is not limited to, the data set forth below in Table 4:

TABLE 4
Patient Diary Data

| Subjective Data | Objective Data |
|---|---|
| Headache Pain Level | Headache Occurrence |
| Sensitivity to Light | Headache Start/End Time |
| Sensitivity to Sound | Acute Medication Usage |
| Aura Before | Rescue Medication Usage |
| Nausea/Vomiting | Medication Start Time |
| Throbbing/Pulsating Pain | Foods/Beverages Ingested Prior |
| Location (Side) - Left/Right/Both | Sleep Pattern |
| Activity/Movement Aleve Pain | Location - Work, Home, etc. |
| Stress Level | Activity - Reading, Computer, etc. |
| Tiredness | |
| Autonomic Symptoms | |

The questions for obtaining the patient diary data can be queried by the RC 50 at times relative to an event or according to a predetermined schedule. For example, when the RC 50 is initially powered on, the patient may be prompted to answer questions regarding headache pain levels, location (side) of the headache pain, acute medications taken, sensitivity to light/sound, the presence of nausea or vomiting, and the presence of autonomic symptoms (e.g., red/tearing eyes, blocked nose, eyelid swelling, etc.). When therapy is stopped, the patient may be prompted to answer questions regarding headache pain. At a predetermined time after therapy stops, such as one hour after therapy, the patient may be prompted to answer questions regarding headache pain levels, rescue medications taken, sensitivity to light/sound, the presence of nausea or vomiting, and the presence of autonomic symptoms.

Additionally, the therapy applied by the medical device 10 may be controlled or otherwise limited or scheduled according to a therapy cycle of a predetermined duration and which includes predetermined intervals according to which therapy is applied. As an example, a therapy cycle may be a 90 minute cycle during which therapy can be applied only during the first 15 minutes. Additionally, prior to beginning therapy, the therapy cycle may require that the headache diary questions be answered. The example therapy cycle may permit continuous or intermittent use during the initial 15 minutes of the 90 minute cycle, and the RC 50 will display the remaining therapy time during the initial 15 minutes of the therapy cycle. Once the 15 minute therapy time expires, no additional therapy is permitted for the remaining 75 minutes of the 90 minute therapy cycle.

According to the invention, the system 100 can facilitate administering stimulation therapy in a post-market setting as a part of an ongoing regimen in combating disorders, such as migraine headaches. The system 100 can also facilitate study of the effectiveness of the stimulation therapy as part of a clinical trial or case study. At the patient level, the physician can program the IMD 20 via the workstation 110 to set the, individualized stimulation parameters, the individualized settings for the RC 50 (e.g., language, diary questions, screen settings, etc.), and any software/firmware updates that may be necessary. The RC 50 then can upload these items to the IMD 20. At home, the patient self-administers, within physician prescribed limits, the stimulation therapy on an as-needed basis, or in accordance with a schedule assigned by a physician/clinician.

The physician WS 110 is outfitted with software that allows the workstation to communicate with the RC 50 when connected thereto via either the wired connection 112 or the wireless connection 114. The physician WS 110 also may communicate with the RC 50 connected remotely to the patient PC 120, via the internet connection 146, 148. The physician WS 110, being additionally connected with the remote database 140, can also access the remote database as a central repository for information relating to clinical trial data for a specific patient and/or other patients enrolled in the trial. Through the remote database 140, administrators, physicians, and clinicians conducting the clinical trial may also transmit stimulation parameters to the RC 50 under the supervision and approval of the patient's physician. If authorized, these settings can then be downloaded to the IMD 20 via the RC 50.

The physician WS 110 can be a PC based system used by the physician to configure the IMDs 20 prior to implantation or post implantation. The programmer (physician) can interface with the RC 50 wirelessly or through the USB connection. In an embodiment, the RC 50 communicates with the physician WS 110 through the wired connection 112, and the controller may enter a pass-through mode in which all or some of the controls are disabled, leaving the controller to simply serves as a communication bridge between the physician WS 110 and the IMD 20. The RC 50 may also communicate with the physician WS wirelessly via the wireless connection 114. Through this communication, the programmer can instruct the RC 50 to communicate with the IMD 20, transmitting and receiving data via their built-in, bi-directional telemetry capabilities. This allows the programming physician to, for example, install or update software/firmware and to set and adjust the stimulation parameters and therapy settings in the IMD 20.

The patient PC 110 can be a PC based system with installed proprietary software that provides for communicating with the RC 50 and relaying data to the remote DB 140. The patient PC 110 is not, however, limited to a PC based system. The system 100 can be adapted to provide for charging and communicating with the RC 50 in a variety of manners. For example, the system 100 may include a standalone charging/docking station with wireless internet communication capabilities for transmitting data to the remote DB 140. In this configuration, a PC is not necessary. As another example, the RC 50 could be fitted with a simple AC power cord for charging and short-range wireless communication capabilities (e.g., Bluetooth) for transmitting data to the remote DB 140 via an external device, such as a Bluetooth enabled PC or cell phone, or a PDA type device.

The remote DB 140 may be built on any platform that allows information to be stored, read, and updated. For example, the remote database can be an industry standard such as Oracle, Microsoft SQL Server, etc., that permits standard SQL (Structured Query Language) commands and queries to store, access, and manipulate the data contained therein. The database can include a table or tables that contain the serial numbers of all IMDs 20 that have been implanted in patients, and can also contain the therapy status of those patients. For example, the database may include all the results of the clinical trial for all patients enrolled in the trial including, but not limited to, the patients' histories, therapy protocol for the patients, therapy efficacy, and treatment regimens for the patients and results to-date. To address privacy concerns, the data stored on the remote DB 140 may be blind to the identity of the patients. The remote DB 140 may, however, may store non-identifying clinically relevant patient data, such as height, weight, blood pressure, sex, and age of the clinical trial participants.

According to one aspect of the invention, the stimulation parameters and therapy settings programmed onto the IMD 20 can include all of the patient and device specific information necessary to perform the stimulation therapy on the patient. It is not necessary to include any patient or therapy specific data (e.g., stimulation parameters/therapy settings) on the RC 50 itself. Due to this, the RC 50 is necessary only to inductively power the IMD 20. This offers a great advantage in that any RC 50, whether it is the patient's personal unit, a physician's unit, or a replacement unit can be used to apply stimulation therapy via the IMD 20 without any pre-programming or set up. The RC 50 may thus be a turn-key unit ready to operate right out of the box.

As another advantage, the RC 50 can also perform its querying and recording functions without any preprogramming either. The RC 50 reads the patient diary questions and schedule from the IMD 20, administers the diary questions, and records the patient diary data accordingly. The RC 50 also reads and records the therapy session data and clinical trial data in real time during the therapy session. The RC 50 thus additionally initiate and administers the patient diary questions, records the therapy session data, and records the patient diary data without pre-programming any patient or trial specific parameters, settings, or data into the RC.

As a further advantage, storing the stimulation parameters and therapy settings on the IMD 20 helps ensure that the therapy will be applied according to the correct patient specific parameters and settings. This also helps ensure that the correct therapy type, patient language, and diary questions are applied/queried to the patient. All of these features advantageously improve the reliability and accuracy of the medical device 10 over a device that includes patient specific settings or parameters on the remote unit.

According to one aspect of the invention, the system 100, and the devices and methods implemented therein, enable a patient to respond to a therapy regimen during a clinical trial and during a post-market usage. In the clinical trial, the stimulation therapy is applied and the patient's responses to the therapy are recorded in an experimental setting according to a clinical trial protocol defined by a clinical trial design so that the efficacy of the therapy can be validated, recorded, studied, and improved.

In a post-market usage, the stimulation therapy is applied as an ongoing treatment regimen tailored by the physician to treat the patient's specific medical condition. The system 100, the medical device 10, and the methods by which the stimulation therapy is applied enable the evaluation of the patient's response by the physician so that the efficacy of that particular patient's treatment regimen can be monitored, adjusted, and improved.

According to the invention, the system 100 can be used to administer a clinical trial that evaluates and tests the effectiveness of the IMD 20 and RC 50 in treating a patient. In the clinical trial setting, the RC 50 and IMD 20 are adapted to apply stimulation therapy according to a clinical trial design that is used to study the effectiveness of the medical device 10 in accordance with established scientific methods of experimentation.

Those skilled in the art will appreciate that there are many different clinical trial designs that may be used to determine the safety and efficacy of the medical device 10, such as a randomized clinical trial design, a blind/double blind clinical trial design, and a placebo-controlled clinical trial design. According to the invention, the medical device 10 can be adapted to conduct a clinical trial conducted in accordance with any of these designs alone or in combination with each other, and with or in combination with any other clinical trial design.

As an example, the system 100 may be used to employ a random insertion of placebo clinical trial design for evaluating the efficacy and safety of the medical device 10. According to this design, the IMD 20 is programmed to apply stimulation therapy to clinical trial subjects/patients in one of several therapy modes, one of which is a placebo mode, that are selected at random and that are unknown (blind design) to the participant. The therapy modes differ from each other in terms of one or more of the stimulation parameters, such as the frequency, duration, amplitude, pulse width, pulse interval, electrode set, and patient limits. In the particular example clinical trial design described herein, there are three therapy modes: full therapy mode, placebo therapy mode, and sub-perception therapy mode.

The full therapy mode applies the full therapeutic stimulation to the subject/patient. In the full therapy mode, the IMD 20 applies stimulation therapy in accordance with the values of the stimulation parameters on the IMD, which are indicated or selected as being the proper values for that particular patient. When the patient receives stimulation therapy in the full therapy mode, the patient receives full therapy and thus can experience all of the sensory perceptions typically associated with receiving this type of (neuro)stimulation. Such sensory perceptions may include, for example, tingling or tickling, sensations commonly associated with neurostimulation. The full therapy mode is the same mode or essentially the same mode that is used as the post clinical trial usage of the system.

The placebo therapy mode applies no stimulation to the subject/patient. Thus, in the placebo therapy mode, the IMD 20 applies no stimulation to the patient. In the placebo therapy mode, the RC 50 may be adapted to provide feedback to the patient indicating that stimulation therapy is being applied. When the patient receives stimulation therapy in the placebo therapy mode, the patient receives no therapy and thus should not experience any of the typically associated sensory perceptions (e.g., tickling, tingling, pain). The placebo therapy mode is transparent to the patient, i.e., in appearance, it is no different that the full therapy mode, expect the patient does not perceive any sensations.

The sub-perception therapy mode applies some therapeutic effect to the subject/patient that is less than that of the full therapy mode. In the sub-perception therapy mode, however, the values for some or all of the stimulation parameters are selected such that the patient experiences no, or substantially no, sensory perceptions (e.g., tickling, tingling,) that would indicate to the patient that full stimulation therapy is being applied. Thus, the sub-perception therapy mode may apply as much stimulation therapy as possible without being perceived by the patient. The use of sub-perception stimulation is that the patient is effectively blinded to whether he/she is receiving placebo or active, albeit non perceived stimulation.

According to the example embodiment of the invention, when the system 100 is used to conduct the clinical trial, the medical device 10 administers stimulation therapy to the trial subject according to a predetermined randomized schedule that dictates which of the therapy modes is applied during any given therapy session. The randomized schedule is part of the therapy settings (see Table 2) stored on the IMD 20. The stimulation parameters for each of the therapy mode is also stored on the IMD 20. The physician can adjust the stimulation parameters for any of the therapy modes.

When the patient initiates a therapy session via the RC 50, the RC retrieves from the IMD 20 the appropriate diary questions based on the therapy type determined by the randomization string. The RC 50 can record the stimulation parameters and therapy settings (see Tables 1 and 2) for the therapy type from the IMD 20. The patient answers the required diary questions, the RC 50 records the patient diary data (see Table 4), and the IMD 20 applies the stimulation therapy according to the therapy mode selected according to the randomized string. As the therapy is applied, the RC 50 records the appropriate therapy session data and clinical trial data (see Tables 3 and 4). Once the stimulated therapy is completed, the patient can complete any necessary patient diary questions. Thereafter, the stimulation parameters, therapy settings, therapy session data, clinical trial data, and patient diary data can be transmitted to the remote DB 140 via the various communication means provided in the system 100. The collected data may remain stored in non-volatile memory of the RC 50 for a predetermined period of time, until more memory registers are needed, until transferred wired or wirelessly to the remote DB 140 via the patient PC 120 or physician WS 110, or the data is erased manually.

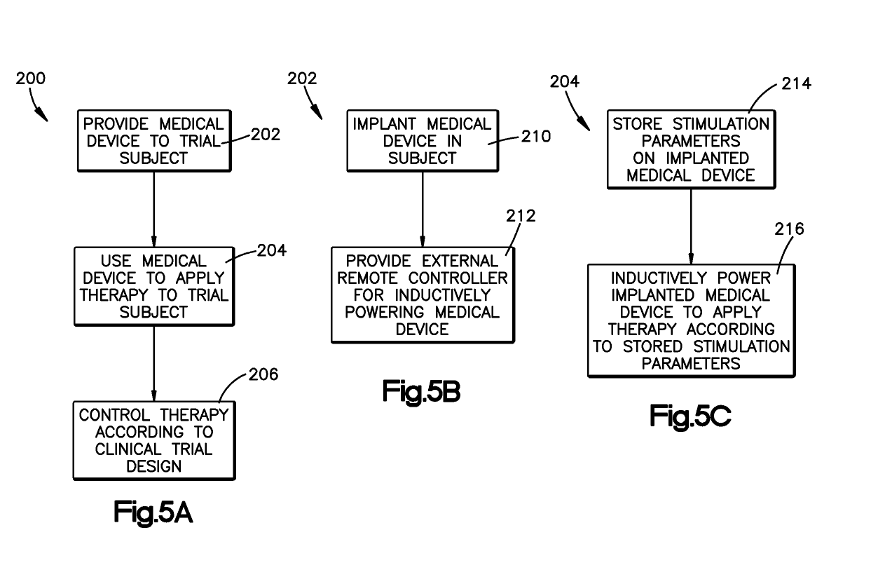

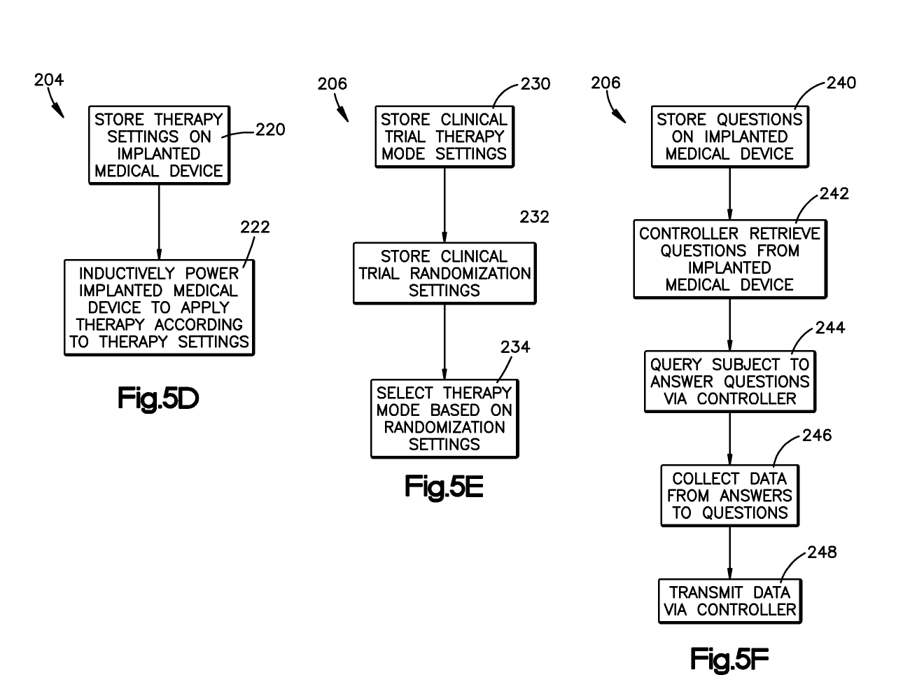

FIGS. 5A-5F illustrate flowchart diagrams of a process 200 by which the system 100 can apply clinical trial therapy to a trial subject. The process 200 illustrated in FIGS. 5A-5F is an example of one process that may be used to administer the clinical trial therapy. Those skilled in the art will appreciate that certain steps in the process may be adjusted, added, omitted, or performed in different order than that illustrated in the figures and described herein without departing from the spirit of the invention. For example, certain steps illustrated and described as being performed in a certain order may be performed simultaneously or in a different order, and certain steps illustrated and described as a single step may comprise multiple steps.

Referring to FIG. 5A, according to the process 200, at step 202, a medical device is provided to the trial subject. The medical device may be provided, for example, by administrators of the clinical trial. At step 204, the medical device is used to apply therapy to the trial subject. Step 204 may, for example, comprise the self-administration of therapy using the medical device. At step 206, the therapy applied to the subject is controlled according to a clinical trial design.

As shown in FIG. 5B, the step 202 of providing a medical device to the trial subject may comprise the step 210 of implanting a medical device, such as the IMD 20, in the trial subject. The IMD 20 may, for example, be an implantable neurostimulator, as described above. The step 202 also may comprise the step of providing a remote controller, such as the RC 50, for providing inductive power to the medical device.

As shown in FIG. 5C, the step 204 of using the medical device to apply therapy to the trial subject may comprise the step 214 of storing stimulation parameters on an implantable medical device, such as the IMD 20 (e.g., an implantable neurostimulator). The stimulation parameters may be parameters such as those set forth in Table 1. The step 204 may also comprise the step 216 of inductively powering the medical device to apply therapy to the subject according to the stimulation parameters.

As shown in FIG. 5D, the step 204 of using the medical device to apply therapy to the trial subject may also comprise the step 220 of storing therapy settings on an implantable medical device, such as the IMD 20 (e.g., an implantable neurostimulator). The therapy settings may be settings such as those set forth in Table 2. The step 204 may also comprise the step 222 of inductively powering the medical device to apply therapy to the subject according to the therapy settings.

As shown in FIG. 5E, the step 206 of controlling therapy applied to the subject according to a clinical trial design may comprise the step 230 of storing clinical trial therapy mode settings on an implantable medical device, such as the IMD 20 (e.g., an implantable neurostimulator). For example, the clinical trial therapy mode settings may be those of the full therapy mode, the placebo mode, and the sub-perception therapy mode described above. For each of these modes, the therapy mode settings may comprise stimulation parameters (e.g., Table 1) and therapy settings (e.g., Table 2). The step 206 may also comprise the step 232 of storing clinical trial randomization settings on the implantable medical device. The randomization settings may, for example, comprise a randomization string. The step 206 may also comprise the step 234 of selecting the clinical trial therapy mode based on the randomization settings.

As shown in FIG. 5F, the step 206 of controlling therapy applied to the subject according to a clinical trial design may comprise the step 240 of storing therapy related questions on an implantable medical device, such as IMD 20 (e.g., an implantable neurostimulator). The therapy related questions may comprise questions designed to elicit from the subject the patient diary data set forth in Table 4. The step 206 may also comprise the step 242 of retrieving via a remote controller (e.g., the RC50) the questions from the implantable medical device. The step 206 may also comprise the step 244 of querying via the remote controller the subject to answer the therapy related questions. The step 206 may comprise the additional step 246 of collecting via the remote controller data in the form of answers to the therapy related questions. The answers may comprise the patient diary data set forth in Table 4. The step 206 may comprise the further step 248 of transmitting via the remote controller the data collected at step 246. This step 248 may, for example, comprise using the RC 50 to transmit the collected data to the remote DB 140.

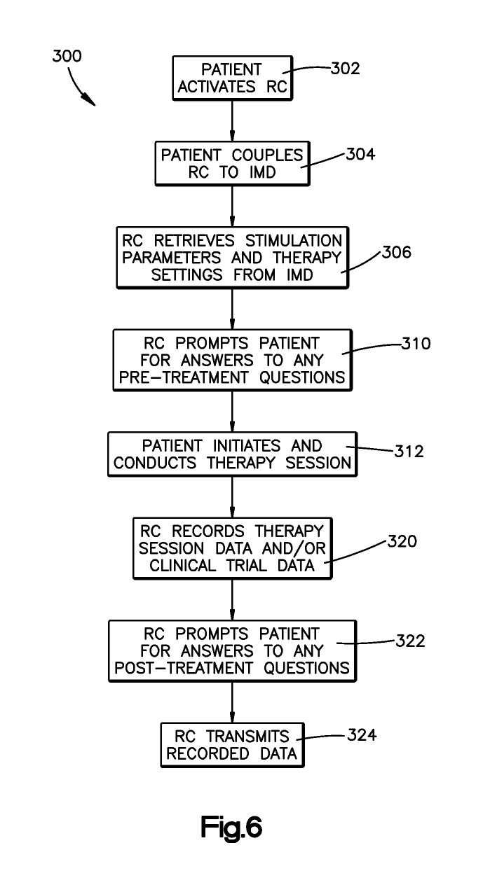

FIG. 6 illustrates a flowchart diagram of a process 300 by which therapy is applied using the medical device 10. The process 300 is illustrative of the method by which a clinical trial subject can apply therapy using the medical device 10 in a clinical trial setting. The process 300 is also illustrative of the method by which a patient can apply therapy using the medical device 10 in a post-market usage of the device. The process 300 illustrated in FIG. 6 is an example of one process that may be used to apply therapy using the medical device 10. Those skilled in the art will appreciate that certain steps in the process may be adjusted, added, omitted, or performed in different order than that illustrated in the figures and described herein without departing from the spirit of the invention. For example, certain steps illustrated and described as being performed in a certain order may be performed simultaneously or in a different order, and certain steps illustrated and described as a single step may comprise multiple steps.

For example, an additional step could be implemented in which the application of therapy described in FIG. 6 may be predicated on gaining approval for using the medical device 10 to apply the therapy. Such approval requirements may be similar or identical to those described in U.S. patent application Ser. No. 12/688,524, filed Jan. 15, 2010, titled “APPROVAL PER USE IMPLANTED NEUROSTIMULATOR,” the disclosure of which is hereby incorporated by reference in its entirety. As another example, in an initial use of an RC 50 with an IMD 20 where the RC has not previously communicated with that particular IMD, the RC may enter an initial mode where the RC prompts the patient with an initial set of predetermined diary questions (e.g., a subset of those set forth in Table 4). Thereafter, the RC 50 will use the patient/clinical trial appropriate questions programmed on the IMD 20.

Referring to FIG. 6, when a patient senses the onset of an event, such as a migraine headache, the patient activates the RC 50, at step 302. The process 300 then proceeds to step 304, where the patient couples the RC 50 to the IMD 30. During coupling step 304, the RC 50 may provide feedback—audible, tactile, or both, that assists the patient in achieving the proper positioning of the RC 50 relative to the IMD 30. Once the RC 50 and IMD 30 are coupled, the process 300 proceeds to step 306, where the RC can retrieve stimulation parameters (Table 1) or therapy settings (Table 2). For example, at step 306, the RC 50 may retrieve patient diary questions to query the patient.

The process 300 then proceeds to step 310, where the RC 50 prompts the patient for answers to any pre-treatment questions. These questions can be designed to elicit from the patient the patient diary data set forth in Table 4. This step 310 may be optional, as it is conditioned on whether the supervising physician/clinician has opted to require pre-treatment questions as a gateway to therapy. The process 300 then proceeds to step 312, where the patient initiates and conducts the therapy session. During the therapy session, if permitted by the supervising physician/clinician, the patient can adjust any adjustable parameters within the physician defined limits. The therapy session ends at step 314 due to either expiration of a timeout period or due to cessation by the patient.

The process 300 proceeds to step 320, where the RC 50 records the therapy session data (Table 3) and/or any necessary clinical trial settings/data (Table 2). The process 300 then proceeds to step 322, where the RC 50 prompts the patient for answers to any post-treatment questions, if the prompting for answers to such questions is enabled by the supervising physician/clinician. At step 342, the IMD 30 records the therapy session data. The process 300 proceeds to step 324, where the RC 50 transmits the recorded data, if this function is enabled. Alternatively, the recorded data may be stored until such a time that transmission of the data is convenient to the patient.

When the RC 50 transmits the recorded data to the remote database, the remote database updates its records and analyzes the data. The database can be utilized to compare the data to pre-programmed data and determines whether an appropriate party, e.g., the patient's physician or an administrator of the clinical trial, should be contacted. If so, the party is contacted and can take the appropriate action, as needed.

From the above description of the invention, those skilled in the art will perceive improvements, changes and modifications. Such improvements, changes and modifications within the skill of the art are intended to be covered by the appended claims.

What is claimed is:

1. A method for conducting a clinical trial with a medical device, the method comprising the steps of:
providing a medical device to a trial subject, the medical device being capable of providing an adjustable level of therapy;
using the medical device to apply therapy to the trial subject; and
controlling the application of therapy applied to the trial subject according to a clinical trial design.

2. The method recited in claim 1, wherein the step of providing a medical device comprises the step of implanting an implantable medical device in the trial subject.

3. The method recited in claim 2, wherein the medical device comprises an implantable neurostimulator device.

4. The method recited in claim 2, wherein the step of using the medical device to apply therapy comprises the step of using a handheld controller to inductively power the implanted medical device and to communicate with the implanted medical device.

5. The method recited in claim 1, wherein the step of controlling the application of therapy comprises the step of selecting a therapy mode, the therapy mode comprising one of a full therapy mode, a placebo therapy mode, and a sub-perception therapy mode.

6. The method recited in claim 5, wherein:
the full therapy mode comprises stimulation parameters for applying to the trial subject providing a predetermined level of stimulation for providing a therapeutic effect to the trial subject;
the placebo therapy mode comprises stimulation parameters for providing no stimulation to the trial subject; and
the sub-perception therapy mode comprises stimulation parameters for applying to the trial subject a predetermined level of stimulation less than the level of stimulation applied in the full therapy mode and that is not perceivable to the patient.

7. The method recited in claim 1, wherein the step of controlling the application of therapy according to the clinical trial design comprises the step of selecting one of a plurality of therapy modes, wherein each therapy mode comprises stimulation parameters that dictate the therapy applied to the trial subjects.

8. The method recited in claim 1, further comprising the steps of:
recording data associated with the therapy applied to the subjects in the group; and
transmitting the recorded data for evaluation.

9. The method recited in claim 1, wherein the clinical trial design comprises a random insertion of placebo clinical trial design, the step of adjusting the level of therapy comprising the step of selecting a therapy mode at random.

10. The method recited in claim 9, wherein the therapy modes comprise stimulation parameters that dictate the therapy applied to the trial subject.

11. The method recited claim 1, wherein the medical device comprises a controller for administering the therapy applied to the trial subject, the method further comprising the step of querying the trial subject to answer questions as a gateway to receiving therapy from the medical device.

12. The method recited claim 1, wherein the medical device comprises a stimulator implantable in the trial subject and a remote controller for controlling operation of the stimulator.

13. The method recited in claim 12, wherein the step of applying therapy comprises the step of using the remote controller to inductively power the implanted stimulator.

14. The method recited in claim 13, wherein the step of applying therapy comprises the step of storing stimulation parameters on the stimulator and using the remote controller to inductively power the stimulator to apply therapy according to the stimulation parameters.

15. The method recited in claim 13, wherein the step of applying therapy comprises the step of storing therapy settings on the stimulator and using the remote controller to inductively power the stimulator to apply therapy according to the therapy settings, the therapy settings comprising clinical trial settings for applying therapy according to the clinical trial design.

16. The method recited in claim 15, wherein the clinical trial settings comprise therapy mode settings and randomization settings.

17. The method recited in claim 16, wherein the therapy mode settings comprise full therapy mode settings, placebo mode settings, and sub-perception therapy mode settings.

18. The method recited in claim 16, wherein the step of adjusting the level of therapy comprises the step of selecting a therapy mode setting based on the randomization setting.

19. The method recited in claim 12, further comprising the steps of:
querying the trial subjects via the remote controller to obtain data in the form of answers to questions related to the therapy;
recording the data on the remote controller; and
transmitting the data via the remote controller for evaluation.

20. The method recited in claim 19, wherein the step of querying the trial subjects comprises the steps of:
storing the questions related to the therapy on the stimulator; and
retrieving via the remote controller the questions from the stimulator.

21. The method recited in claim 19, wherein the questions related to the therapy are predetermined so that the data comprises subjective data and objective data.

22. An apparatus for applying stimulation therapy to a subject in a clinical trial, the apparatus comprising:
an implantable medical device; and
a remote controller for inductively powering the medical device and communicating with the medical device, the implantable medical device being adapted to store therapy settings and simulation parameters for applying the stimulation therapy, the remote controller being operative to record therapy data during the application of stimulation therapy.

23. The apparatus recited in claim 22, wherein the implantable medical device is capable of providing an adjustable level of stimulation therapy, the settings and parameters being selected to control the application of stimulation therapy applied to the subject according to a clinical trial design.

24. The apparatus recited in claim 22, wherein the medical device comprises an implantable neurostimulator device.

25. The apparatus recited in claim 23, wherein the therapy settings and stimulation parameters define a plurality of clinical trial therapy modes comprising:
a full therapy mode comprising stimulation parameters for applying to the subject a predetermined level of stimulation therapy for providing a therapeutic effect to the trial subject;
a placebo therapy mode comprising stimulation parameters for providing placebo therapy to the trial subject; and
a sub-perception therapy mode comprising stimulation parameters for applying to the trial subject a predetermined level of stimulation therapy less than the level of stimulation therapy applied in the full therapy mode and that is not perceivable to the patient.

26. The apparatus recited in claim 22, wherein the remote controller is operative to transmit the recorded therapy data for evaluation.

27. The apparatus recited in claim 22, wherein the remote controller is operative to query the trial subject to answer questions as a gateway to providing the stimulation therapy.

28. The apparatus recited in claim 22, wherein the medical device stores stimulation parameters on the medical device and the remote controller inductively powers the medical device to apply the stimulation therapy according to the stimulation parameters.

29. The apparatus recited in claim 22, wherein the medical device stores therapy settings on the medical device and the remote controller inductively powers the medical device to apply the stimulation therapy according to the therapy settings, the therapy settings comprising clinical trial settings for applying therapy according to a clinical trial design.

30. The apparatus recited in claim 29, wherein the clinical trial settings comprise therapy mode settings and randomization settings.

31. The apparatus recited in claim 30, wherein the medical device is operative to select a therapy mode setting based on the randomization setting.

32. The apparatus recited in claim 22, wherein the remote controller is operative to:
query the trial subjects to obtain data in the form of answers to questions related to the stimulation therapy;
record the data on the remote controller; and
transmit the data via the remote controller for evaluation.

33. The apparatus recited in claim 32, wherein the implantable medical device is operative to store the questions related to the therapy and the remote controller is operative to retrieve the questions from medical device.

34. The apparatus recited in claim 32, wherein the questions related to the stimulation therapy are predetermined so that the data comprises subjective data and objective data.

35. A system for conducting a clinical trial on a medical device, the system comprising:
an implantable medical device for applying stimulation therapy;
a remote controller for inductively powering the medical device and communicating with the medical device, the implantable medical device being adapted to store therapy settings and simulation parameters for applying the stimulation therapy, the remote controller being operative to record therapy data during the application of stimulation therapy; and
a communication network over which the remote controller can transmit the recorded therapy data in order to conduct the clinical trial.

36. The system recited in claim 35, further comprising a patient personal computer operatively connected to the communication network, the patient personal computer being operative to communicate with the remote controller via the communication network.

37. The system recited in claim 35, further comprising a physician workstation operatively connected to the communication network, the physician workstation being operative to communicate with the remote controller via the communication network.

38. The system recited in claim 35, further comprising a remote database operatively connected to the communication network, the remote database being operative to communicate with the remote controller via the communication network.

39. The system recited in claim 35, further comprising:
a patient personal computer operatively connected to the communication network;
a physician workstation operatively connected to the communication network; and
a remote database operatively connected to the communication network, wherein the patient personal computer, the physician workstation, the remote database, and the remote controller are operative to communicate with each other via the communication network.

40. The system recited in claim 35, wherein the implantable medical device is capable of providing an adjustable level of stimulation therapy, the settings and parameters being selected to control the application of stimulation therapy applied to the subject according to a clinical trial design.

41. The system recited in claim 35, wherein the medical device comprises an implantable neurostimulator device.

42. The system recited in claim 35, wherein the therapy settings and stimulation parameters define a plurality of clinical trial therapy modes comprising:
a full therapy mode comprising stimulation parameters for applying to the subject a predetermined level of stimulation therapy for providing a therapeutic effect to the trial subject;
a placebo therapy mode comprising stimulation parameters for providing placebo therapy to the trial subject; and
a sub-perception therapy mode comprising stimulation parameters for applying to the trial subject a predetermined level of stimulation therapy less than the level of stimulation therapy applied in the full therapy mode and that is not perceivable to the patient.

43. The system recited in claim 35, wherein the medical device stores stimulation parameters on the medical device and the remote controller inductively powers the medical device to apply the stimulation therapy according to the stimulation parameters.

44. The system recited in claim 35, wherein the medical device stores therapy settings on the medical device and the remote controller inductively powers the medical device to apply the stimulation therapy according to the therapy settings, the therapy settings comprising clinical trial settings for applying therapy according to a clinical trial design.

45. The system recited in claim 44, wherein the clinical trial settings comprise therapy mode settings and randomization settings.

46. The system recited in claim 45, wherein the medical device is operative to select a therapy mode setting based on the randomization setting.

47. The system recited in claim 35, wherein the remote controller is operative to:
query the trial subjects to obtain data in the form of answers to questions related to the stimulation therapy;
record the data on the remote controller; and
transmit the data over the communication network to a remote database for evaluation.

48. The system recited in claim 47, wherein the implantable medical device is operative to store the questions related to the therapy and the remote controller is operative to retrieve the questions from medical device.

49. The system recited in claim 47, wherein the questions related to the stimulation therapy are predetermined so that the data comprises subjective data and objective data.
